(19) United States
(12) Patent Application Publication
(10) Pub. No.: US 2020/0241016 A1
(43) Pub. Date: Jul. 30, 2020

(54) ENDOCRINE DISRUPTOR FIELD TESTER

(71) Applicant: IONTOX, LLC, KALAMAZOO, MI (US)

(72) Inventor: James McKim, Kalamazoo, MI (US)

(73) Assignee: IONTOX, LLC, KALAMAZOO, MI (US)

(21) Appl. No.: 16/777,821

(22) Filed: Jan. 30, 2020

Related U.S. Application Data
(63) Continuation of application No. 15/274,913, filed on Sep. 23, 2016, now abandoned.
(60) Provisional application No. 62/232,553, filed on Sep. 25, 2015.

(51) Int. Cl. G01N 33/74; G01N 33/543

(52) U.S. Cl. CPC G01N 33/74; G01N 33/743; G01N 33/54366

(57) ABSTRACT

The IONTOX endocrine field tester (EFT) provides an easy method for determining the presence, type, and amount of endocrine active chemicals. By developing a bead/receptor/column/reagent for only the target hormones, it is possible to detect a broad range of endocrine active materials for each receptor type. The EFT kit will provide the first simple method for monitoring environmental water systems that can be done in the field. Similar columns can also be prepared for androgens and for thyroid hormones. This means that the same technology can be applied to several different endocrine targets and the market and revenue significantly expanded.

### CROSS-REFERENCE TO RELATED APPLICATIONS

This application is a continuation of U.S. patent application Ser. No. 15/274,913 filed Sep. 26, 2016, which claims benefit of U.S. Provisional Application No. 62/232,553, filed Sep. 25, 2015, the contents of which is hereby incorporated in its entirety including all tables, figures and claims.

### FIELD OF THE DISCLOSURE

This disclosure pertains to methods and apparatuses for detecting low, medium, and high concentrations of endocrine disruptors in field samples or biological samples.

### BACKGROUND OF THE DISCLOSURE

A major concern for many manufacturing facilities in the United States and Europe is the monitoring of effluent water, soil, and air for endocrine active compounds or other chemical classes. The U.S. EPC endocrine disruptor program requires companies to screen their products and company waste streams for the presence of endocrine disruptors (each an “ED”). An ED is any chemical that mimics an endogenous substance by binding to a specific receptor.

Currently, there is not a simple field test to quantitatively determine whether there are chemicals that act like estrogens, androgens, thyroid hormones, other hormones, or chemicals of other classes. Currently, effluent samples must be collected, labeled, and analyzed by expensive LC/MS analytical procedures.

### SUMMARY OF THE DISCLOSURE

The IONTOX endocrine field tester (EFT) provides an easy method for determining the presence, type, and amount of endocrine active chemicals. The method can employ a small tubular column filled with beads to which are attached hormone receptors. A vial containing hormone that specifically binds to the receptors is then eluted through the column. As the fluid moves down the column hormone binds to its receptor and is held on the beads. Following this loading step, a sample (effluent) is passed through the column. Chemicals present in the effluent that can bind to the receptor will displace the receptor-bound hormones. As the effluent is washed through the column it is collected in a sample reagent bottle. The volume added and collected is recorded. Effluent containing the target hormone (e.g. estrogen) is contacted with a reagent that changes color, phosphoresces, fluoresces or otherwise indicates the presence of hormone. The amount of hormone displaced can be quantified and is directly related to the amount of endocrine disruptor contaminant in the test sample. In this way it is possible to determine the presence and/or amount of endocrine disruptors in the test sample. There are hundreds of chemicals that mimic body hormones. By developing a bead/receptor/column/reagent for only the target hormones, it is possible to detect a broad range of endocrine active materials for each receptor type. The entire process can be completed in less than 30 minutes. The EFT kit will provide the first simple method for monitoring environmental water systems that can be done in the field. Similar columns can also be prepared for androgens and for thyroid hormones. This means that the same technology can be applied to several different endocrine targets and the market and revenue significantly expanded.

### BRIEF DESCRIPTION OF THE DRAWINGS

FIG. 1 is a front elevational view of a packed column that can be incorporated into the endocrine disruptor field test kits and used in the methods of detecting or quantifying low concentrations of endocrine disruptors in samples. Note, the diameter and length of columns can change to optimize detection. This is accomplished by changing the surface area in the column. Higher surface area for test samples with low concentrations allows for the column to function by concentrating analytes.

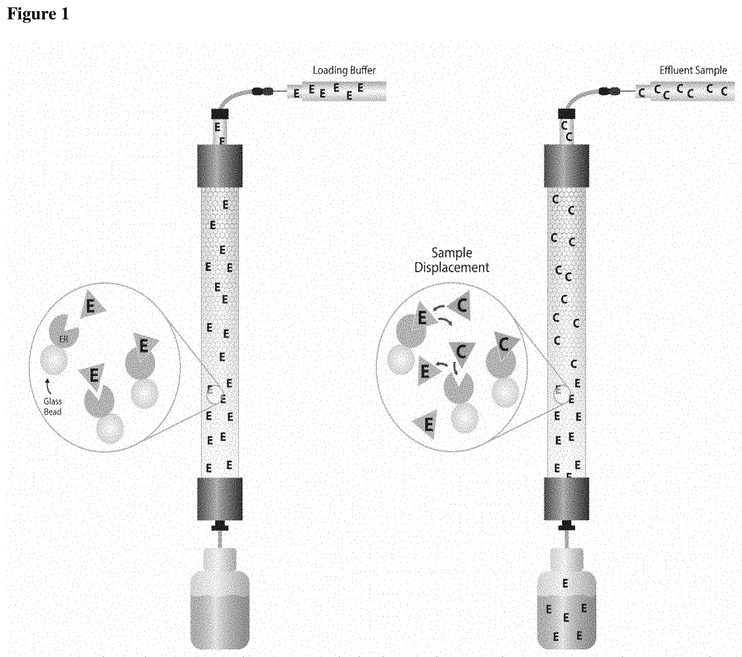

### DETAILED DESCRIPTION

Endocrine Disruptors is defined as any chemical that mimics a natural substance by binding to a specific receptor.

The term “hormone receptor” as used herein refers to a natural hormone receptor or synthetically produced materials that have substantially the same functionality of a natural hormone receptor, such as recombinant hormone receptors. Techniques for obtaining natural hormone receptors and synthesizing recombinant hormone receptors are well known. Such receptors are also commercially available.

The term “bound” means that the hormone receptor is physically or chemically attached to the carrier, such as by covalent, ionic or hydrogen bonding, so that negligible amounts of the hormone receptor are displaced from the carrier when a sample that is free of endocrine disruptor is passed through or over the carrier material. This term also refers to the binding of hormones or endocrine disruptors to the hormone receptors.

The carrier can be comprised of any solid substrate material to which a hormone receptor can be bound, including sepharose, glass, ceramics, plastics, or paper. The carrier can be in the form of beads (e.g., spherical beads), pellets, or rings that can be randomly or structurally packed. Techniques of binding a receptor to a substrate are well known. Such techniques include occlusion or physical confinement, physical absorption, ionic bonding and covalent bonding, including multipoint covalent bonding.

The indicator reagent is a chemical compound that is capable of interacting with hormones to facilitate detection of hormones displaced from the carrier due to the presence of endocrine disruptors in the sample. The indicator reagent can be a chemical that changes color upon interaction with the hormone, or a chemical that phosphoresces or fluoresces when it interacts with the hormone. Instruments such as photosensors, can be used to measure the intensity of a color change, phosphorescence or fluorescence. The intensity of the signal can be correlated to the amount of hormone in the effluent or detection zone, which can in turn be correlated to the concentration of endocrine disruptor in the sample.

It is envisioned that as an alternative to a packed column format, the test kit could be provided in a test strip format in which a sample is deposited or applied to a sample application zone of a test strip, and drawn or wicked into an adjacent carrier that has bound hormone receptors that are pre-loaded with (i.e., bound to) hormones. Adjacently downstream of the carrier is a detection zone that can have an indicator reagent that reacts with any hormones displaced by endocrine disruptors in the sample to provide a color change or other indication that can be quantified, either visually or with instruments.

Disclosed is a method of determining the identity and quantity of endocrine disruptors in water, soil, or air or biological samples such as blood or urine.

Human or animal estrogen receptor can be bound to a bead and packed into a column. Any receptor can be attached to the bead so the method encompasses all receptors. Receptors may be recombinant or isolated from animal or human tissues. Once bound to the bead, the receptor is loaded with its natural ligand (e.g., estrogen is eluted through the column, where it binds to the estrogen receptors already attached to the beads). Once the column receptors are loaded with their ligand, sample water, soil extracts, biological samples or air may be pumped through the column. The presence of chemicals capable of binding to the receptor is detected because the natural receptor ligand (e.g., estrogen) is displaced by the competing chemical and eluted from the column. The reagents are designed to detect the presence of estrogen or any other hormone by causing a color change, or producing a fluorescence, radiometric, or luminescence signal.

The column in which beads are packed can be made of glass, plastic, or any other material.

Recombinant receptors for human or animal can be prepared using standard molecular biology techniques. Each receptor type (e.g. testosterone, thyroid, or progesterone to name only a few) can be bound to an unique column.

The eluted estrogen or any other receptor ligand (e.g., testosterone, thyroid hormone, progesterone etc.) can be detected by collecting the eluent containing displaced hormone in a bottle containing reagents that provide a color change in the presence of the target ligand (e.g., estrogen). The intensity of color signal is related to the amount of chemical in the test sample. Variations of this method may include other signal endpoints such as fluorescence, radioisotopes, antibody linked detection or luminescence.

Columns may be prepared of varying lengths, and diameters. The capacity of binding and sample detection is dependent on or related to column size. The material in the columns may be sepharose beads, glass beads or any other relevant material.

Also disclosed is a kit encompassing the column packed or unpacked with beads to which target receptors have been attached, a syringe to move or add sample, the loading buffer, and the reagents necessary for detection of target chemical. The column may also be attached to a pump that would transfer sample water (e.g. from a lake) through the column.

The testing may also be done as a service.

We envisage that this method may also be used to detect other chemical classes. Receptors may be replaced with a protein to which a chemical will bind, or an antibody.

The IONTOX Endocrine Field Tester (“EFT”) comprises a kit and an easy method for determining the presence, type, and amount of endocrine active chemicals or other chemical classes in water, soil, air or in biological matrices such as blood or urine.

The kit can be comprised of the following apparatuses and chemicals, used in combination:

1. One or more small, tubular columns packed with beads or pellets to which are attached human recombinant hormone receptors (for example, but without limitation, estrogen). The beads or pellets may be supported within the column by wire mesh or another supporting material. Columns may be prepared of varying lengths, and diameters. The capacity of binding and sample detection is dependent on or related to column size. The beads or pellets are made of sepharose, glass, plastic, ceramic or any other material suitable for attaching human recombinant hormone receptors. The recombinant receptors for human or animal are prepared using known molecular biology techniques.

2. One or more loading buffers or vials containing estrogen, progesterone, testosterone, thyroid hormone, other receptor ligands, or chemical of other classes.

3. One or more syringes to move or add effluent.

4. Reagents necessary for detection of the target ligand(s) or chemical(s).

5. One or more receptacles for receiving effluent after it passes through the column(s).

The method can be comprised of the following steps:

1. A bead-preparation step wherein one or more human or animal hormone receptors is bound to a set of beads and packed into a column (one hormone receptor per column). Any receptor can be attached to the bead so the method encompasses all receptors. Receptors may be recombinant or isolated from animal or human tissues. Receptors may be replaced with a protein to which a chemical will bind, or an antibody.

2. A loading step wherein one or more of receptor ligands (for example, but without limitation, estrogen) is eluted through a corresponding column. As the fluid moves down the column, the ligand binds to its receptor and is held on the beads or pellets. By binding to the bead, the receptor is loaded with its natural ligand (e.g., estrogen is eluted through the column, where it binds to the estrogen receptors already attached to the beads).

3. A sampling step wherein sample water, soil extracts, air, or other effluents are passed or pumped through the column. Chemicals present in the effluent that are active with respect to the loaded ligand(s) (for example, but without limitation, estrogen) will displace the ligand that was bound to the receptors in step 2.

4. A detection step wherein effluent that has passed through a column is collected into a vial containing one or more reagents that turn color, luminesce, fluoresce, or otherwise detectably react in the presence of the loaded ligand. The presence of chemicals capable of binding to the receptor is detected because the natural receptor ligand (e.g., estrogen) is displaced by the competing chemical and eluted from the column. The eluted estrogen or other receptor ligand (e.g., testosterone, thyroid hormone, progesterone, etc.) is detected by collecting the eluent in a receptacle containing reagents that provide a color change in the presence of the target ligand (e.g., estrogen). Variations of this method may include other signal endpoints such as fluorescence, radioisotopes, antibody-linked detection, or luminescence.

5. A quantification step wherein the intensity of the color signal or other signal produced in step 4 is used to determine the amount of target ligand in the test sample.

6. A correlation step wherein the amount of ligand eluted is used to calculate the amount of contaminant in the effluent. In this way it is possible to determine the presence of estrogen or other target ligands, active chemicals, and the amount thereof.

The entire process can be completed in less than 30 minutes.

The EFT kit will provide the first simple method for monitoring environmental water systems that can be done in the field. Similar columns can also be prepared for androgens and for thyroid hormones. This means that the same technology can be applied to several different endocrine targets and the market and revenue significantly expanded.

The described embodiment is preferred and/or illustrated, but is not limiting. Various modifications are considered within the purview and scope of the appended claims.

What is claimed is:

1. An analysis kit or device for detecting and/or quantifying the presence or amount of an endocrine disruptor in a sample, comprising:
a carrier defining a flow path for the sample;
a sample inlet at one end of the flow path and a detection zone or effluent outlet at another end of the flow path;
hormone receptors bound to the carrier;
hormones bound to the hormone receptors or held in a container for application to the carrier and binding to the hormone receptors prior to introduction of the sample to the sample inlet; and
a reagent that indicates the presence or amount of the endocrine disruptor in the sample at the detection zone or effluent outlet.
